(19) United States
(12) Patent Application Publication
(10) Pub. No.: US 2006/0141071 A1
(43) Pub. Date: Jun. 29, 2006

(54) Compositions and methods for treating gynecological disorders

(75) Inventor: Steven Walter Clavey, Victoria (AU)

Correspondence Address: CLARK & ELBING LLP, 101 FEDERAL STREET, BOSTON, MA, 02110, US

(73) Assignee: Chinese Medicines Scientific Consultants Pty Ltd, North Sydney, New South Wales (AU)

(21) Appl. No.: 10/562,195

(22) PCT Filed: Jun. 27, 2003
(86) PCT No.: PCT/AU03/00820

§ 371 Date: Dec. 23, 2005

(51) Int. Cl. A61K 36/254; A61K 36/736; A61K 36/28; A61K 36/65

(52) U.S. Cl. 424/728; 424/735; 424/764; 424/769; 424/773; 424/779

(57) ABSTRACT

A composition for treating or alleviating a symptom resulting from a gynaecological disorder, comprising the herbs Chi Shao (Paeonia obova, Root), Dang Gui (Angelica Polymorpha, Root), Chuan Xiong (Ligusticum Wallichii, Root), Xiang Fu (Cyperus Rotundus, Rhizome), Yan Hu Suo (Corydalis turtschanninovii, Rhizome), Tao Ren (prunus persica, Seed) or parts thereof.

### TECHNICAL FIELD

This invention relates to new medicinal compositions and methods for treating gynaecological disorders, in particular the treatment or prevention of Endometriosis, Adenomyosis or Dysmenorrhoea.

### BACKGROUND

Any discussion of the prior art throughout the specification should in no way be considered as an admission that such prior art is widely known or forms part of common general knowledge in the field.

Gynaecological disorders such as endometriosis involve the presence of endometrial cells (i.e. the cells lining the uterus) outside of the lumen of the uterus, at other sites within the cavity of the pelvis; or in the case of ademomyosis, within the muscle wall of the uterus itself. These fragments of abnormally located tissue (‘endometriotic lesions’) pass through the same periodic changes as the uterine lining, but lack an outlet for the bleeding that occurs from them. The presence of free blood within the pelvic cavity or within the muscle wall of the uterus can lead to severe pain and numerous complications such as the formation of adhesions or lesions, increase in size and bulk of the uterus, and stricture of fallopian tubes or bowel.

The current treatment is surgical followed by menstrual suppression, now predominantly via the contraceptive pill.

Other gynaecological disorders such Secondary Dysmenorrhea can be related to the presence of these pelvic lesions, secondary to gynaecological disorders such as endometriosis, pelvic inflammatory disease and other related conditions. Similarly, Primary Dysmenorrhea is not related to any pelvic lesion, but is one of the most common gynaecological complaints.

It has now surprisingly been found that the formulations of the present invention ameliorate at least some of the disadvantages of the prior art therapies and methods, or at least provide useful alternatives.

### SUMMARY OF THE INVENTION

The formulations of the present invention are designed to provide a treatment and/or prevention for recurrent Endometriosis or Adenomyosis, and/or alleviate the symptoms of recurrent Endometriosis, Adenomyosis or Dysmenorrhoea. In particular, the present invention can alleviate pelvic discomfort following several surgical interventions for Endometriosis or Adenomyosis; especially when such intervention has resulted in increased discomfort for the patient.

Accordingly, in a first aspect, the present invention provides a composition comprising the herbs:

| | |
|---|---|
| Chi Shao | (Paeonia obovata, Root) |
| Dang Gui | (Angelica Polymorpha, Root) |
| Chuan Xiong | (Ligusticum Wallichii, Root) |
| Xiang Fu | (Cyperus Rotundus, Rhizome) |
| Yan Hu Suo | (Corydalis turtschaninovii, Rhizome) |
| Tao Ren | (Prunus persica, Seed), or parts thereof. |

According to a second aspect, the present invention provides a composition comprising the herbs Paeonia obovata, Ligusticum wallichii, Angelica Polymorpha, Cyperus Rotundus, Corydalis turtschaninovii, Prunus persica, Poria cocos, Alisma plantago-aquatica, Atractylodes macrocephala, Spatholobus suberectus, Lindera Strychnofolia, Glycyrrhiza uralensis, or parts thereof.

The compositions set out above may be supplemented with one or more herbs selected from the group consisting of:

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |
| Fang Feng | (Ledebouriella seseloides, root) |
| Chen Pi | (Citrus reticulata, fruit pericarp) |
| Chai Hu | (Bupleurum falcatum, root) |
| Xu Duan | (Dipsacus asper, root) |
| Ye Jiao Teng | (Polygonum Multiflorum, vine stem) |
| Tu Si Zi | (Cuscuta, Seed) |
| Shan Zha | (Crataegus cuneata, fruit) |
| He Huan Pi | (Albizziae julibrissin, bark) |
| Yi Mu Cao | (Leonurus sibiricus, Herb) |
| Tian Qi | (Panax pseudoginseng, Root) |
| Hong Hua | (Carthamus tinctorius, Flos) |
| Sang Ji Sheng | (Loranthus parasiticus, Stem) |
| Xiao Hui Xiang | (Foeniculum Vulgare, Fruit) |
| Gui Zhi | (Cinnamomum cassia, stem), or parts thereof. |

The above supplementary herbs may be conveniently grouped into subsets, and as such added to the compositions of the first or second aspect, for treatment of particular conditions. The preferred subsets are as follows:

Subset a: ‘Endometriosis with blood deficiency and poor immunity’

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |

Subset b: ‘Endometriosis affecting the bowels’

| | |
|---|---|
| Fang Feng | (Ledebouriella seseloides, root) |
| Chen Pi | (Citrus reticulata, fruit pericarp) |
| Shan Zha | (Crataegus cuneata, fruit) |

Subset C: ‘Endometriosis with anxiety’

| | |
|---|---|
| Chai Hu | (Bupleurum falcatum, root) |
| Ye Jiao Teng | (Polygonum multiflorum, vine stem) |
| He Huan Pi | (Albizziae julibrissin, bark) |

Subset d: ‘Endometriosis with lower back ache’

| | |
|---|---|
| Xu Duan | (Dipsacus asper, root) |
| Tu Si Zi | (Cuscuta, Seed) |
| Sang Ji Sheng | (Loranthus parasiticus, Stem) |

Subset e: ‘Endometriosis with severe menstrual clotting and/or pelvic congestion’

| | |
|---|---|
| Yi Mu Cao | (Leonurus sibiricus, Herb) |
| Tian Qi | (Panax pseudoginseng, Root) |
| Hong Hua | (Carthamus tinctorius, Flos) |

Subset f: ‘Endometriosis with coldpain’

| | |
|---|---|
| Xiao Hui Xiang | (Foeniculum Vulgare, Fruit) |
| Gui Zhi | (Cinnamomum cassia, stem) |

It will be understood however, that the conditions identified for treatment with formulations comprising the various subsets of herbs need not be as designated above. The designations are provided merely as an example of preferred groupings and possible indications of use.

Preferably, the compositions are formulated with dried herbs which can be decocted for use by the patient. Herbs may also be in powdered form. However, the compositions of the present invention can also be formulated with aqueous, organic or aqueous/organic extracts of the herbs, as would be understood by those skilled in the art, which extracts may be dried and formulated by conventional means. The composition may also be delivered in a capsule or tablet dosage form, but it can also be delivered in a liquid or paste form. The composition may further include pharmaceutically acceptable excipients, adjuvants, solvents, disintegrators, carriers, flavours, colourings or coatings (suitable excipients, adjuvants, solvents, carriers, flavours, colourings and coatings as found in standard texts such as for example Remington: The Science and Practice of Pharmacy, 19^{th} Ed., Mack Publishing Co., 1995, incorporated in its entirety herein by reference).

According to a third aspect, the present invention provides a method of therapeutic or prophylactic treatment of a gynaecological disorder comprising the step of administering to a subject requiring such treatment, a composition according to the first or second aspect.

According to a fourth aspect, the present invention provides a method of alleviating a symptom resulting from a gynaecological disorder, comprising the step of administering to a subject requiring such treatment, a composition according to the first or second aspect.

According to a fifth aspect, the present invention provides a method of therapeutic or prophylactic treatment of endometriosis, adenomyosis or dysmenorrhoea comprising the step of administering to a subject requiring such treatment, a composition according to the first or second aspect.

According to a sixth aspect, the present invention provides a method of alleviating a symptom resulting from endometriosis, adenomyosis or dysmenorrhoea comprising the step of administering to a subject requiring such treatment, a composition according to the first or second aspect.

It is preferable that the treatment is administered orally. The treatment may be administered in a single bolus dose, multiple doses or via a slow release device.

It will be appreciated that the treatment may be administered in conjunction with other therapeutic agents.

According to a seventh aspect, the present invention provides a composition according to the first or second aspect for use as a medicament.

According to an eighth aspect, the present invention provides the use of a composition according to the first or the second aspect for the manufacture of a medicament for prophylactic or therapeutic treatment of a gynaecological disorder.

According to a ninth aspect, the present invention provides the use of a composition according to the first or the second aspect for the manufacture of a medicament for prophylactic or therapeutic treatment of endometriosis, adenomyosis or dysmenorrhoea.

It will be understood by those skilled in the art that the formulations of the present invention may be suitably used, and be effective in the treatment not only of Endometriosis but also of related conditions, such as Adenomyosis or Dysmenorrhoea. Hence, when referring to the preferred “Endometriosis formulation”, it is intended that such a formulation also applies to Adenomyosis or Dysmenorrhoea.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### DETAILED DESCRIPTION OF THE INVENTION

The treatment of medical disorders using herbal formulations differs from orthodox Western-style treatments in that the herbal formulations are intended to be tailored to meet the specific requirements of each patient. The invention provides a base or core formulation which may be adjusted to treat endometriosis and/or adenomyosis or general disturbances in the menstrual cycle typified by PMS and/or dysmenorrhoea and the resulting symptoms which may manifest in a patient suffering these conditions. Moreover, the formulation of the invention is more gentle than what has been available previously or is currently available, enhances healing and recovery from surgery and is particularly beneficial for patients that react poorly to surgical intervention for endometriosis and/or adenomyosis.

The components of a core formulation comprising the herbs

| | |
|---|---|
| Chi Shao | (Paeonia obovata, Root) |
| Dang Gui | (Angelica Polymorpha, Root) |
| Chuan Xiong | (Ligusticum Wallichii, Root) |
| Xiang Fu | (Cyperus Rotundus, Rhizome) |
| Yan Hu Suo | (Corydalis turtschaninovii, Rhizome) |
| Tao Ren | (Prunus persica, Seed), or parts thereof. |

may be supplemented with one or more additional herbs selected from the group consisting of:

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |
| Fang Feng | (Ledebouriella seseloides, root) |
| Chen Pi | (Citrus reticulata, fruit pericarp) |
| Chai Hu | (Bupleurum falcatum, root) |
| Xu Duan | (Dipsacus asper, root) |
| Ye Jiao Teng | (Polygonum Multiflorum, vine stem) |
| Tu Si Zi | (Cuscuta, Seed) |
| Shan Zha | (Crataegus cuneata, fruit) |
| He Huan Pi | (Albizziae julibrissin, bark) |
| Yi Mu Cao | (Leonurus sibiricus, Herb) |
| Tian Qi | (Panax pseudoginseng, Root) |
| Hong Hua | (Carthamus tinctorius, Flos) |
| Sang Ji Sheng | (Loranthus parasiticus, Stem) |
| Xiao Hui Xiang | (Foeniculum Vulgare, Fruit) |
| Gui Zhi | (Cinnamomum cassia, stem), or parts thereof. |

The above supplementary herbs may be conveniently grouped into subsets, and as such added to compositions comprising the herbs:

| | |
|---|---|
| Chi Shao | (Paeonia obovata, Root) |
| Dang Gui | (Angelica Polymorpha, Root) |
| Chuan Xiong | (Ligusticum Wallichii, Root) |
| Xiang Fu | (Cyperus Rotundus, Rhizome) |
| Yan Hu Suo | (Corydalis turtschaninovii, Rhizome) |
| Tao Ren | (Prunus persica, Seed), or parts thereof. |

or comprising the herbs:
Paeonia obovata, Ligusticum wallichii, Angelica Polymorpha, Cyperus Rotundus, Corydalis turtschaninovii, Prunus persica, Poria cocos, Alisma plantago-aquatica, Atractylodes macrocephala, Spatholobus suberectus, Lindera Strychnofolia, Glycyrrhiza uralensis, or parts thereof.

Specific tailored formulations, comprising the core and additional supplementary herbs, which may usefully be prescribed for treatment, are also included as individual embodiments of the present invention.

Subset a: ‘Endometriosis with blood deficiency and poor immunity’

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |

Subset b: ‘Endometriosis affecting the bowels’

| | |
|---|---|
| Fang Feng | (Ledebouriella seseloides, root) |
| Chen Pi | (Citrus reticulata, fruit pericarp) |
| Shan Zha | (Crataegus cuneata, fruit) |

Subset C: ‘Endometriosis with anxiety’

| | |
|---|---|
| Chai Hu | (Bupleurum falcatum, root) |
| Ye Jiao Teng | (Polygonum multiflorum, vine stem) |
| He Huan Pi | (Albizziae julibrissin, bark) |

Subset d: ‘Endometriosis with lower back ache’

| | |
|---|---|
| Xu Duan | (Dipsacus asper, root) |
| Tu Si Zi | (Cuscuta, Seed) |
| Sang Ji Sheng | (Loranthus parasiticus, Stem) |

Subset e: ‘Endometriosis with severe menstrual clotting and/or pelvic congestion’

| | |
|---|---|
| Yi Mu Cao | (Leonurus sibiricus, Herb) |
| Tian Qi | (Panax pseudoginseng, Root) |
| Hong Hua | (Carthamus tinctorius, Flos) |

Subset f: ‘Endometriosis with cold pain’

| | |
|---|---|
| Xiao Hui Xiang | (Foeniculum Vulgare, Fruit) |
| Gui Zhi | (Cinnamomum cassia, stem) |

One such formulation described herein in Example 1 is the preferred formulation (also referred to as the “endometriosis formulation”).

Further formulations for treating Endometriosis and other gynaecological conditions with specific symptom-patterns, eg ‘Endometriosis pain with lower back discomfort’ or ‘Endometriosis irritating the bowel’, can be created using the core and supplementary components described herein.

Thus, the supplementary herbs are not crucial to the treatment, but provide significant advantages. For convenience, and without limitation, the supplementary herbs may be grouped (see examples). Typically, one to six more herbs may be chosen from the additional set, depending on the patient's symptoms and needs of the treatment.

The orally or parenterally administered embodiments of the herbal formulation of this invention can be in any conventional form such as example capsules, tablets, elixirs, powders, granules, suspensions in water or non-aqueous media and sachets as additives to food or beverages.

The herbal formulation may be produced utilizing concentrated herbal extract, either an extract of the total formulation, or by combining separate extracts of individual herbs. Such extracts may be employed as the base materials in the manufacture of tablets or powders.

In powder form, the various dry powdered ingredients are mixed together until a substantially homogeneous mixture is obtained. The powder can be administered by mixing with a liquid, such as water or juice for consumption of the resulting suspension. Liquid preparations may take the form of, for example, solutions, syrups or suspensions or they may be presented as a dry powder for reconstitution with an appropriate liquid carrier, preferably water, for oral administration or for packaging in containers for example in ampoules or in multi-dose containers. Such liquid preparations may be prepared by conventional means with pharmaceutically acceptable additives such as suspending agents, emulsifying agents, preservatives, non-aqueous vehicles example almond oil, oily esters or ethyl alcohol and artificial or natural colours and/or sweeteners. The composition may also be delivered in a capsule or tablet dosage form, but it can also be delivered in a liquid or paste form. In tablet form, the ingredients are mixed together and the tablets prepared according to methods well known in the art, for example the wet granulation method, the dry-granulation method or direct compression. See Remington: The Science and Practice of Pharmacy, 19^{th} Ed., Mack Publishing Co., 1995, incorporated in its entirety herein by reference.

Tablets or solid dosage forms containing the active substances with or without suitable additives are prepared either by compression or moulding methods well known in the art. The tablets may be uncoated, or may be coated by known techniques to delay disintegration and absorption in the gastrointestinal tract and thereby provide a sustained action over a longer period. For example, a time-delay material such as glyceryl monostearate or glyceryl distearate may be employed. Coating may also be performed using techniques described in U.S. Pat. Nos. 4,256,108; 4,160,452; and 4,265,874 to form osmotic therapeutic tablets for controlled release.

Formulations for oral use may be in the form of hard gelatin capsules in which the active ingredients are mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

The composition may further include pharmaceutically acceptable excipients, adjuvants, solvents, disintegrators, carriers, flavours, colourings or coatings (suitable excipients, adjuvants, solvents, carriers, flavours, colourings and coatings as found in standard texts such as for example Remington's Pharmaceutical Sciences).

Animals or humans may be treated in accordance with the invention. The present invention is also directed to methods of treating or alleviating the symptoms associated with a gynaecological disorder, for example, endometriosis, adenomyosis or dysmenorrhea.

Generally, the terms “treating”, “treatrnent” and the like are used herein to mean affecting a subject, tissue or cell to obtain a desired pharmacological and/or physiological effect. The effect may be prophylactic in terms of completely or partially preventing a gynaecological disorder or sign or symptom thereof, and/or may be therapeutic in terms of a partial or complete cure of a disorder. “Treating” as used herein covers any therapy of, or prevention of, a gynaecological disorder in a mammal, particularly a human, and includes preventing the disorder from occurring in a subject who may be predisposed to the disorder, but has not yet been diagnosed as having it; inhibiting the disorder, i.e. arresting its development; or relieving or ameliorating the effects of the disorder i.e. cause regression of the effects of the disorder.

The herbal formulation according to the invention may be administered orally or parenterally in a therapeutically effective dose. The herbal formulation is preferably administered orally and in dosage units. It will be appreciated that the formulation may be administered in a single bolus dose, multiple doses or via a slow release device.

For example, sustained and/or timed release formulations may be made by sustained release means or delivery devices that are well known to those of ordinary skill in the art, such as those described in U.S. Pat. Nos. 3,916,899, 3,536,809, 3,598,123, 4,008,719, 4,710,384, 5,674,533, 5,059,595, 5,591,767, 5,120,548, 5,073,543, 5,639,476, 5,354,556 and 5,733,566, the disclosures of which are each incorporated herein by reference. These compositions can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the herbal compositions of the invention. Thus, single unit dosage forms suitable for oral administration, such as, but not limited to, tablets, capsules, gelcaps, caplets, powders, and the like, that are adapted for sustained release are encompassed by the present invention. As described above, all known methods for encapsulation which are compatible with the properties of the herb formulations are encompassed by this invention.

For treatment of a subject, depending on activity of the herbal formulation, manner of administration, nature and severity of the disorder, age and body weight of the subject, different daily doses can be used. Under certain circumstances, however, higher or lower doses may be appropriate. The administration of the daily dose can be carried out both by single administration in the form of an individual dose unit or else several smaller dose units and also by multiple administration of subdivided doses at specific intervals.

A preferred dose for treating or preventing Endometriosis, Adenomyosis or Dysmenorrhea is in the range of 5 g -30 g (equivalent raw herb dosage) per day. However, the exact dose depends to a great extent on the severity of the gynaecological disorder and the health of the patient. More preferably, 5 g -10 g (equivalent raw herb dosage) is administered both morning and night; but if administered in a single dose, 10 g (equivalent raw herb dosage) is preferred.

Preferred embodiments of the invention will now be described by way of example only with reference to the following case studies.

### EXAMPLES

### Example 1

### Formulations and Preparation

The components of a core set of herb useful in the preparation of the formulations of the present invention are listed in Table 1 below. Omission of one or more of the core set may make the formulation less effective in the treatment, but would still exhibit the advantages of the present invention.

TABLE 1
Core herbs

| Chinese name | Pharmaceutical name |
|---|---|
| Chi Shao | (Paeonia obovata, Root) |
| Dang Gui | (Angelica Polymorpha, Root) |
| Chuan Xiong | (Ligusticum Wallichii, Root) |
| Xiang Fu | (Cyperus Rotundus, Rhizome) |
| Yan Hu Suo | (Corydalis turtschaninovii, Rhizome) |
| Tao Ren | (Prunus persica, Seed) |

The most preferred formulation for treatment of Endometriosis, Adenomyosis or Dysmenorrhea is set out in Table 2 below:

TABLE 2
Preferred formulation (“Endometriosis formulation”)

| Chinese | | |
|---|---|---|
| name | Pharmaceutical name | amounts (grams) |
| Chi Shao | Paeonia obovata, root | 300 |
| Bai Shao | Paeonia leaf, flora, Radix | 300 |
| Fu Ling | Poria cocos, sclerotium | 200 |
| Ze Xie | Alisma plantago-aquaticae, rhizome | 200 |
| Dang Gui | Angelica polymorpha, root | 120 |
| Bai Zhu | Atractylodes macrocephala, rhizome | 150 |
| Chuan Xiong | Ligusticum wallichii, root | 120 |
| Xiang Fu | Cyperus rotundus, rhizome | 150 |
| Yan Hu Suo | Corydalis turtschaninovii, rhizome | 150 |
| Tao Ren | Prunus persica, seed | 120 |
| Ji Xue Teng | (Spatholobus suberectus, root & stem) | 300 |
| Wu Yao | (Lindera strychnofolia, root) | 150 |
| Gan Cao | (Glycyrrhiza uralensis, root) | 60 |

Chi Shao can be replaced, in total or partially, by Bai Shao (Paeonia lactiflora, root). Bai Shao is simply the cultivated form of Chi Shao. Ji Xue Teng (Spatholobus suberectus, root & stem) could be replaced by Hong Teng (Sargentodoxa cuneata, vine stem).

The dried herbs were macerated, then percolated for 21 days in a solution of water and 97% ethanol (35 litres water, 15 litres ethanol, resulting in a 25% alcoholic tincture). After percolation, the liquid was drained and strained over a period of 2 days, giving a final liquid volume of approximately 35 litres. The product description is provided below.

Product Description

Colour: Translucent amber liquid
Odour: Aromatic; sweet; penetrating; reminiscent of Angelica
Taste and flavour: Sharp, aromatic; faintly acrid, characteristic of Angelica 100-150 ml (depending upon the patient's digestive strength) was then decanted and consumed in the morning and again in the evening.

In the preferred formulations the only herb prepared beyond the raw stage was Yan Hu Suo (Corydalis turtschaninovii, rhizome), which was soaked in vinegar (for example undiluted rice vinegar) overnight, then baked dry in an oven at about 180° until thoroughly dry. This increased the availability of the alkaloid corydaline and thus improved the analgesic effect.

### Example 2

### Extraction Method

The volatile oils were extracted by water and steam distillation from:

| | |
|---|---|
| Dang Gui | Angelica polymorpha, root |
| Xiang Fu | Cyperus rotundus, rhizome |
| Chuan Xiong | Ligusticum wallichii, root |

Although it can be appreciated that fresh herbs can be used, in this instance, the dried herbs were ground and then covered with a layer of water in a container having a steam inlet. The oily layer or volatile oils of the condensed distillate was then separated from the aqueous layer. This allows the oils to be preserved and not evaporated during the decoctions steps that follow. The remaining macerated herbal mixture was then combined with the other desired herbs and the whole formula was decocted (boiled in water) twice. The first decoction used nine times the amount of water to the weight of the raw herbs, to allow for absorption into the dried herb material, to decoct for 1.5 hours. The second decoction used somewhat less water, (i.e. seven times the amount of water to weight of the dried herbs) to decoct for 1 hour. The resulting liquids that remained after boiling were, mixed and filtered and then concentrated at low heat till 1:15. The resulting liquid was filtered again, spray-dried and the resulting dried particles then sprayed with the volatile oils extracted earlier, and tested to meet the required specifications.

The resultant formulation was a brownish-tan powder with minute speckles of brown and white and had an odour of earthy wood with Angelica.

Certain additional herbs, which can be used in conjunction with the core set, or in conjunction with the preferred formulation, are listed in Table 3 below. One or more additional herbs, typically one to six additional herbs, selected from those listed in Table 3, assist in addressing the symptoms peculiar to the individual patient. The additions are appropriate in prescriptions customized for individual patients.

TABLE 3
Preferred additional herbs.

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |
| Fang Feng | (Ledebouriella seseloides, root) |
| Chen Pi | (Citrus reticulata, fruit pericarp) |
| Chai Hu | (Bupleurum falcatum, root) |
| Xu Duan | (Dipsacus asper, root) |
| Ye Jiao Teng | (Polygonum Multiflorum, vine stem) |
| Tu Si Zi | (Cuscuta, Seed) |
| Shan Zha | (Crataegus cuneata, fruit) |
| He Huan Pi | (Albizziae julibrissin, bark) |
| Yi Mu Cao | (Leonurus sibiricus, Herb) |
| Tian Qi | (Panax pseudoginseng, Root) |
| Hong Hua | (Carthamus tinctorius, Flos) |
| Sang Ji Sheng | (Loranthus parasiticus, Stem) |
| Xiao Hui Xiang | (Foeniculum Vulgare, Fruit) |
| Gui Zhi | (Cinnamomum cassia, stem) |

One or more herbs from the above list were used to create further “Endometriosis” formulations, depending on which additional symptoms needed treatment. Not wishing to be bound by any theory or proposed mechanisms of action, the herbs set out in Table 3 may be conveniently grouped into sub-sets for specific indications.

Subset a: ‘Endometriosis with blood deficiency and poor immunity’

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |

Subset b: ‘Endometriosis affecting the bowels’

| | |
|---|---|
| Fang Feng | (Ledebouriella seseloides, root) |
| Chen Pi | (Citrus reticulata, fruit pericarp) |
| Shan Zha | (Crataegus cuneata, fruit) |

Subset C: ‘Endometriosis with anxiety’

| | |
|---|---|
| Chai Hu | (Bupleurum falcatum, root) |
| Ye Jiao Teng | (Polygonum multiflorum, vine stem) |
| He Huan Pi | (Albizziae julibrissin, bark) |

Subset d: ‘Endometriosis with lower back ache’

| | |
|---|---|
| Xu Duan | (Dipsacus asper, root) |
| Tu Si Zi | (Cuscuta, Seed) |
| Sang Ji Sheng | (Loranthus parasiticus, Stem) |

Subset e: ‘Endometriosis with severe menstrual clotting and/or pelvic congestion’

| | |
|---|---|
| Yi Mu Cao | (Leonurus sibiricus, Herb) |
| Tian Qi | (Panax pseudoginseng, Root) |
| Hong Hua | (Carthamus tinctorius, Flos) |

Subset f: ‘Endometriosis with coldpain’

| | |
|---|---|
| Xiao Hui Xiang | (Foeniculum Vulgare, Fruit) |
| Gui Zhi | (Cinnamomum cassia, stem) |

### Example 3

### Tabletting Process

Tablets were prepared by adding to the core herbs and one or more supplementary herbs, 100 mg calcium phosphate, 135 mg cellulose and 200 mg cross carmellose. The mixture was blended for 10 minutes and milled using a Fitz mill having a 60 mesh screen size. The milled particles had a particle size distribution of less than 5 wt % of the particles being greater than a 60 mesh screen size.

20 mg silica was added to the milled formulation and blended for a further 15 minutes to form a dry blend. 10 mg magnesium stearate was then added to the dry blend and blended for a further 5 minutes.

Tablets were then prepared using a Killian Tablet Press. The tablets were evaluated for theoretical weight (950 mg), hardness (17 Kp) and friability (less than 1%).

### Example 4

### The tablets prepared in Example 3 were placed in a Freund HiCoater HCF 150 film coating machine and slowly rotated and preheated for approximately 15 minutes to 35 C.

While the tablets were preheated, the coating mixture was prepared by adding 15 litres of cold water to 1.5 kg hydroxy propyl methyl cellulose in a 20 litre stainless steel container. The ingredients were stirred until completely dissolved. Once dissolved, the coating pan holding the tablets was rotated at 5 RPM and the tablets were spray coated under exhaust until a coating weight gain of 1.5 to 2% was achieved. The coated tablets were then removed from the coating pan and packed.

### Example 5

### Case Study 1.

Patient 1 was 23 years of age. At presentation she had suffered with Endometriosis for two years during which time she had undergone two laparoscopies with laser treatment for extensive and severe endometriotic lesions. She was prescribed Danocrine from which she suffered dizziness, weight gain, hot flushing and thirst. As a result of the Endometriosis she suffered lower abdominal stabbing pain in the lower right quadrant, and fluid retention. The initial prescription considered both the Endometriosis-associated pain and the side effects of the medication.

Prescription 1

| Chinese name | Pharmaceutical name | Dried Herb |
|---|---|---|
| Fu Ling | Poria cocos, Sclerotium | 12 g |
| Ze Xie | Alisma plantago-aquatica, Rhizome | 15 g |
| Dang Gui | Angelica polymorpha, Radix | 12 g |
| Bai Zhu | Atractylodes macrocephala, Rhizoma | 12 g |
| Xiang Fu | Cyperus rotundus, Rhizome | 12 g |
| Yan Hu Suo | Corydalis turtschaninovii, Rhizome | 12 g |
| Gan Cao | Glycyrrhizae uralensis, Radix | 3 g |
| He Huan Pi | Albizzia julibrissin, bark | 15 g |
| Yu Jin | Curcumae, Tuber | 12 g |
| Ren Dong Teng | Lonicerae Japonicae, Ramus | 18 g |
| Chuan Lian Zi | Meliae Toosendan, Fructus | 12 g |
| Fo Shou Pian | Citrii Sarcodactylis, Fructus | 6 g |
| Ban Xia | Pinelliae Ternata, Rhizoma | 12 g |
| Shi Chang Pu | Acori Graminei, Rhizoma | 12 g |
| Shen Qu | Massa Fermentata | 15 g |

Dosage: 100 mls of decoction in the morning and evening.
Second Consultation

At the second consultation two weeks later, the patient reported a reduction in the side-effects of the medication Danocrine, and less severe Endometriosis-associated pain. Based on these observations the prescription was customized further for her specific requirements. She was advised to take prescription 2.

Prescription 2

| Chinese name | Pharmaceutical name | Dried Herb |
|---|---|---|
| Chi Shao | Paeonia obovata, Radix | 9 g |
| Bai Shao | Paeonia lactiflora, Radix | 9 g |
| Fu Ling | Poria cocos, Sclerotium | 15 g |
| Ze Xie | Alisma plantago-aquatica, Rhizoma | 12 g |
| Dang Gui | Angelica polymorpha, Radix | 12 g |
| Bai Zhu | Atractylodes macrocephala, Rhizome | 15 g |
| Chuan Xiong | Ligusticum wallichii, Radix | 12 g |
| Xiang Fu | Cyperus rotundus, Rhizoma | 12 g |
| Yan Hu Suo | Corydalis turtschaninovii, Rhizoma | 2 g |
| Gan Cao | Glycyrrhiza uralensis, Radix | 3 g |
| He Huan Pi | Albizzia julibrissin, bark | 15 g |
| Chai Hu | Bupleurum falcatum, Root | 6 g |
| Chen Pi | Citrus Reticulata, pericarpium | 6 g |
| Fo Shou Pian | Citrii Sarcodactylis, Fructus | 6 g |
| Chuan Lian Zi | Meliae Toosendan, Fructus | 9 g |
| Hong Teng | Caulis Sargentodoxae Cuneatae | 18 g |
| Gui Zhi | Cinnamomi Cassiae, Ramulus | 9 g |

Dosage as for above (ie. 100 mls of decoction morning and evening)
Third Consultation.

At the third consultation four weeks later the patient reported feeling very well and the disappearance of abdominal pain apart from occasional slight twinges. She was able to stop taking Danocrine and had not taken it for three weeks leading up to the consultation. She was advised to repeat administration of prescription 2.

Fourth Consultation.

Two months later she obtained a repeat prescription. She reported only one day of severe pain which was the second day of her last period.

Fifth Consultation.

Ten months later she called and reported that she had been feeling exceptionally well. She had taken the contraceptive pill for 6 months and noticed some pain after discontinuing use. She was advised to take a repeat of prescription 2.

At a follow-up consultation she reported that she had not suffered any endometriosis-related pain. Ultrasound scans found no trace of residual Endometriosis. Twelve months of treatment with the herbal formulation had successfully resolved her condition.

### Example 6

### Case Study 2

Patient 2 was 30 years of age. Endometriosis had been diagnosed 5 years prior to presentation. She had undergone a laparoscopy, fallen pregnant and had not had a problem until recently at which she reported having Endometriosis pain from ‘umbilicus to toes’. The pain was worse at night than during the day. She was advised to take the following prescription:

| Chinese name | Pharmaceutical name | Dried Herb |
|---|---|---|
| Chi Shao | Paeonia obovata, Radix | 9 g |
| Bai Shao | Paeonia lactiflora, Radix | 9 g |
| Dang Gui | Angelica polymorpha, Radix | 9 g |
| Chuan Xiong | Ligusticum wallichii, Radix | 9 g |
| Xiang Fu | Cyperus rotundus, Rhizoma | 12 g |
| Yan Hu Suo | Corydalis turtschaninovii, Rhizoma | 9 g |
| Tao Ren | Prunus persica, Semen | 9 g |
| Ji Xue Teng | Spatholobus suberectus, Radix et Caulis | 15 g |
| Ye Jiao Teng | Polygonum multiflorum, vine stem | 30 g |
| He Huan Pi | Albizzia julibrissin, bark | 18 g |
| Chai Hu | Bupleurum falcatum, Root | 6 g |
| Sheng Di | Rehmannia glutinosa, Radix | 15 g |
| Fo Shou Pian | Citrii Sarcodactylis, Fructus | 6 g |
| Mu Dan Pi | Moutan Radicis, Cortex | 9 g |
| San Leng | Sparganii, Rhizoma | 9 g |
| E Zhu | Curcumae Zedoariae, Rhizoma | 9 g |

Dosage: 150 mls decoction morning and evening.

Usually a smaller dose is given if there is a suspicion that a patient may have trouble with the taste of the decoction. This is usually not a problem with the tablets or capsules made from extracts. In case of tablet, capsule and the like formulations, the dosage can be standardised.

Second Consultation.

At the second consultation four months later four months later, the patient reported that she had not experienced any Endometriosis pain since taking the prescription. She reported having some nausea and headaches prior to her periods, as she had historically always had.

Third Consultation.

The third consultation took place thirteen months later for a reason unrelated to Endometriosis. She did report that she had only had mild Endometriosis symptoms on 3 occasions in the time since the second consultation.

### Example 7

### Case Study 3

Patient 3 was 24 years of age. At presentation Endometriosis had been diagnosed two months previously. Her symptoms included primary dysmenorrhoea involving the abdomen, thighs and lower back, dyspareunia, abdominal bloating and distension everyday, as well as hot flushes, thirst and some palpitations. Menarche was at 11 years of age. Her menstrual cycle was typically 21 days and her period lasted 6-7 days with medium heavy bleeding, preceded by fluid retention and increased abdominal bloating. Her pulse was deep, slippery and rapid. Her tongue had a thin coating and a red tip. She was advised to take the following prescription:

Prescription 1

| Chinese name | Pharmaceutical name | Dried Herb |
|---|---|---|
| Chi Shao | Paeonia obovata, Radix | 9 g |
| Bai Shao | Paeonia lactiflora, Radix | 9 g |
| Fu Ling | Poria cocos, Sclerotium | 15 g |
| Ze Xie | Alisma plantago-aquatica, Rhizoma | 15 g |
| Dang Gui | Angelica polymorpha, Radix | 12 g |
| Bai Zhu | Atractylodes macrocephala, Rhizoma | 12 g |
| Chuan Xiong | Ligusticum wallichii, Radix | 9 g |
| Xiang Fu | Cyperus rotundus, Rhizoma | 12 g |
| Yan Hu Suo | Corydalis turtschaninovii, Rhizoma | 12 g |
| Tao Ren | Prunus persica, Semen | 9 g |
| Ji Xue Teng | Spatholobus suberectus, Radix | 12 g |
| | et Caulis | |
| Wu Yao | Lindera strychnofolia, Radix | 12 g |
| Gan Cao | Glycyrrhizae uralensis, Radix | 3 g |
| Shen Qu | Massa Fermentata | 15 g |
| Xiao Hui Xiang | Foeniculi Vulgaris, Fructus | 3 g |
| Da Huang | Rhei, Rhizoma | 9 g |

Dosage: 120 mls of decoction in the morning and evening.

This patient was provided with 3 bags of herbs. (Each of the herbs in the list above is measured out at the weight specified and placed in a bag for the patient to take home and decoct. One bag usually provides enough herbal liquid to last 3-4 days at the dosage indicated.).

Second Consultation.

At the second consultation the patient reported that she experienced no Endometriosis pain with her last period. Also, the abdominal bloating was reduced with administration of the prescription. She reported still being thirsty, but experienced fewer palpitations. Her pulse was deep and rapid, and the front of her tongue red. She was advised to take the same prescription again.

Third Consultation.

The third consultation took place three weeks later. She reported feeling well despite not taking the prescription. She was advised to continue taking the prescription.

Fourth Consultation.

The fourth consultation took place six months later. The patient reported having no endometriosis pain for several months but then her pain with the period and with intercourse gradually returned. The patient required further consolidation of her condition and was advised to take prescription 2.

Prescription 2

| Chinese name | Pharmaceutical name | Dried Herb |
|---|---|---|
| Bai Shao | Paeonia lactiflora, Radix | 9 g |
| Chi Shao | Paeonia obovata, Radix | 9 g |
| Fu Ling | Poria cocos, Sclerotium | 15 g |
| Ze Xie | Alisma plantago-aquatica, Rhizoma | 15 g |
| Dang Gui | Angelica polymorpha, Radix | 12 g |
| Xiang Fu | Cyperus rotundus, Rhizoma | 12 g |
| Yan Hu Suo | Corydalis turtschaninovii, Rhizoma | 12 g |
| Ji Xue Teng | Spatholobus suberectus, Radix et Caulis | 15 g |
| Wu Yao | Lindera strychnofolia, Radix | 2 g |
| Gan Cao | Glycyrrhiza Uralensis, Radix | 3 g |
| Da Huang | Rhei, Rhizoma | 9 g |
| Shen Qu | Massa Fermentata | 15 g |
| Mu Xiang | Saussureae seu Vladimiriae, Radix | 12 g |
| Xiao Hui Xiang | Foeniculi Vulgaris, Fructus | 3 g |

### Example 8

### Case Study 4.

Patient 4 was 40 years of age. She had suffered painful periods for three years and eight months. Endometriosis was diagnosed by laparoscopy at 6 months from pain commencing. Her doctor informed her that it was extensive. Laser treatment followed to remove the Endometriosis lesions. The sustained symptoms included pain in waves, every ten minutes during her periods, which were heavy with dark red clotting. Her menstrual cycle was 24-25 days and her period lasted 4-5 days. Her period was preceded by breast distension, fluid retention and headache. She also complained of chest ache in the cold, and lower backache at times. Her pulse was deep, slippery and somewhat rapid and weak at the chi position. Her tongue was slightly red and thinly coated. Her immediate concern was the chest ache and rib pain for which she was prescribed a prescription specifically for this purpose.

Second Consultation.

The second consultation was three months later. The patient reported that she was no-longer experiencing chest and rib pain. Her endometriosis pain was, however, at the umbilical level, and she described it as a sharp intermittent twisting-like gripping. She reported that the pain improved with the application of heat and was worse when sitting down. She also experienced lumbar pain. She reported reduced PMS symptoms except that her breasts were still sore. She was advised to take the following prescription which was formulated to treat Endometriosis:

Prescription 1

| Chinese name | Pharmaceutical name | Dried Herb |
|---|---|---|
| Chi Shao | Paeonia obovata, Radix | 9 g |
| Bai Shao | Paeonia lactiflora, Radix | 9 g |
| Fu Ling | Poriae cocos, Sclerotium | 18 g |
| Dang Gui | Angelica polymorpha, Radix | 12 g |
| Chuan Xiong | Ligusticum wallichii, Radix | 9 g |
| Xiang Fu | Cyperus rotundus, Rhizoma | 12 g |
| Yan Hu Suo | Corydalis turtschaninovii, Rhizoma | 12 g |
| Tao Ren | Prunus persica, Semen | 9 g |
| Ji Xue Teng | Spatholobus suberectus, Radix et Caulis | 12 g |
| Wu Yao | Lindera strychnofolia, Radix | 15 g |
| Gan Cao | Glycyrrhiza Uralensis, Radix | 3 g |
| He Huan Pi | Albizziae julibrissin, bark | 15 g |
| Chuan Duan | Dipsacus asper, root | 12 g |
| Sang Ji Sheng | (Loranthus parasiticus, Stem) | 15 g |
| Ba Ji Tian | Morindae Officianalis, Radix | 12 g |

Dosage: 150 mls of decoction morning and evening.
Third Consultation.

The third consultation was 10 weeks later. The patient reported that she experienced only a twinge of discomfort, less clotting and few PMS symptoms with her last two periods. She also reported the absence of any ordinary period pain. The patient was advised to repeat the prescription.

Fourth Consultation.

The fourth consultation was 7 weeks later. The patient reported only slight discomfort with her last period. Her lower back was still painful and she was still experiencing premenstrual breast distension. She was advised to repeat the prescription.

Fifth Consultation.

The fifth consultation was 8 weeks later. The patient reported no pain with her last period. Her lower back had improved and her breasts were still slightly sore. Nineteen months after the fifth consultation the patient reported that she was feeling very well and that she was not experiencing any Endometriosis pain at all. Six months of treatment with the herbal formulation had successfully resolved her condition.

### Example 9

### Case Study 5

Patient 1 was 45 years of age. At presentation she had suffered with Adenomyosis (endometriosis within the body of the uterus) for four years during which time she had undergone a laparotomy and ‘debulking’ of uterus (removal of adenomyosis) subsequent to suffering two miscarriages. Surgery improved her symptoms by 80% initially, but in the three years since the surgery the symptoms were gradually returning.

At presentation, as a result of the adenomyosis there was dyspareunia (painful intercourse), the uterus felt swollen and hard, and menstrual bleeding was excessively heavy, with bleeding also occurring at mid-cycle (ovulation). There was abdominal distention and emotional distress involving palpitations, worsening premenstrually.

Prescription 1

| Chinese name | Pharmaceutical name | Herb Dosage |
|---|---|---|
| Chi Shao | Paeonia Rubra, Radix | 20 g |
| Yan Hu Suo | Corydalis Yanhusuo, Rhizoma | 12 g |
| Fu Ling | Poriae Cocos, Sclerotium | 12 g |
| Ji Xue Teng | Spatholobus Suberectus, Radix | 30 g |
| Ze Xie | Alismatis Plantago-aquaticae, Rhizoma | 12 g |
| He Huan Pi | Albizziae Julibrissin, Cortex | 15 g |
| Tao Ren | Prunus Persica, Semen | 9 g |
| Dang Gui | Angelica Polymorpha, Radix | 12 g |
| Wu Yao | Lindera Strychnofolia, Radix | 12 g |
| Bai Zhu | Atractylodis Macrocephalae, Rhizoma | 12 g |
| Chuan Xiong | Ligustici Wallichii, Radix | 12 g |
| Xiang Fu | Cyperi Rotundi, Rhizoma | 12 g |
| Ye Jiao Teng | Polygonum Multiflorum, vine stem | 30 g |
| Qian Cao Gen | Rubia Cordifolia, Radix | 12 g |
| Mu Gua | Chaenomeles Lagenaria, Fructus | 12 g |

Dosage: 150 mls of decoction in the morning and evening.

The final two herbs were added for the excessively heavy bleeding. The remaining herbs are in the preferred formulation or a subset.

Second Consultation

At the second consultation one month later, the patient reported a marked reduction in the pain, abdominal distention, emotional distress, and palpitations. In her words, she ‘felt great!’ There was much less bleeding at ovulation—‘only two spots’, and the menstrual bleeding was reduced with more even flow. Based on these observations the prescription was repeated, with the addition of a single herb for dryness of the eyes:
Gou Qi Zi Lyciurn chinensis, fruit 12 g (from subset for blood deficiency)
   Third Consultation

At the third consultation four weeks later the patient reported that the uterus had become swollen and hard for one week at midcycle (ovulation), although less than before initial presentation, and there was mild emotional upset prior to the menstruation. She was advised to repeat administration of prescription 1.

Fourth Consultation

One month later the patient reported that there had been no pain with the period, with less heavy bleeding. Her premenstrual symptoms were better ‘by far’, with better sleep and less distress. Again the initial prescription was repeated.

Fifth Consultation.

Two months later she reported that she had been feeling exceptionally well. There was much less abdominal distention, the menstruation was ‘really good’ and the bleeding back within normal ranges.

At a follow-up consultation for another condition some months later, she reported that she had not suffered any adenomyosis pain, periods were less heavy, and menstruation was stable. Five months of treatment with the herbal formulation had successfully resolved her condition.

### Example 10

### Case Study 6

Patient 2 was 39 years of age. Endometriosis had been diagnosed 16 years prior to presentation. She had undergone two laparoscopies, 10 years apart, the most recent 5 years before her initial presentation for herbs. The surgeon had reported that the endometriosis was restricted in area but substantial, and also involved pelvic adhesions.

On presentation the patient reported that in the previous six weeks she had noticed a recurrence of the symptoms she had had prior to the surgery 5 years before. This involved a bilateral abdominal stabbing pain that persisted, constantly present from midcycle to the period, which was also painful with heavy bleeding and a dragging sensation in the abdomen. Her lower back had also become painful. Generally she felt cold, with abdominal distention and borborygmus. She was advised to take the following prescription:

Prescription 1

| Chinese name | Pharmaceutical name | Herb dosage |
|---|---|---|
| Chi Shao | Paeonia Obovata, Radix | 9 g |
| Bai Shao | Paeonia Lactiflora, Radix | 9 g |
| Dang Gui | Angelica Polymorpha, Radix | 9 g |
| Chuan Xiong | Ligusticum Wallichii, Radix | 9 g |
| Xiang Fu | Cyperus Rotundus, Rhizoma | 12 g |
| Yan Hu Suo | Corydalis Turtschaninovii, Rhizoma | 12 g |
| Fu Ling | Poriae Cocos, Sclerotium | 12 g |
| Gan Cao | Glycyrrhiza Uralensis, Radix | 3 g |
| Chai Hu | Bupleurum Falcatum, Radix | 6 g |
| He Huan Pi | Albizzia Julibrissin, Pericarpium | 15 g |
| Xu Duan | Dipsacus Asper, Radix | 12 g |
| Gui Zhi | Cinnamomum Cassia, Ramulus | 9 g |
| Chen Pi | Citrus Reticulata, Fruit Pericarp | 9 g |

The last five herbs are from the subsets, the first two Chai Hu and He Huan Pi=subset ‘Endo with anxiety’, then Xu Duan =subset ‘Endo with lower backache’, Gui Zhi=subset ‘Endo with cold pain’, and Chen Pi=subset ‘Endo affecting the bowels’.

Dosage: 150 mls decoction morning and evening. The patient was also prescribed a tincture for Endometriosis comprised of the preferred formulation (see Specifications: ‘tincture’). This tincture was to be taken when the decocted herbs were finished, 2 mls in water, AM and PM.

Second Consultation

At the second consultation two months later, the patient reported that she had had ‘almost instant relief for the constant pain’, less dragging, and little lower backache. She still, however, suffered from occasional intense bilateral ‘hot’pain that increased with bowel movements. Prior to periods, she was less cold and had no dragging pain, but there remained some considerable dysmenorrhea associated with clotting and discoloured menstrual blood. Based on these observations the prescription was customized further for her specific requirements. She was advised to take prescription 2.

Prescription 2

| Chinese name | Pharmaceutical name | Herb dosage |
|---|---|---|
| Chi Shao | Paeonia Obovata, Radix | 12 g |
| Bai Shao | Paeonia Lactiflora, Radix | 12 g |
| Dang Gui | Angelica Polymorpha, Radix | 12 g |
| Chuan Xiong | Ligusticum Wallichii, Radix | 9 g |
| Xiang Fu | Cyperus Rotundus, Rhizoma | 12 g |
| Yan Hu Suo | Corydalis Turtschaninovii, Rhizoma | 12 g |
| Fu Ling | Poriae Cocos, Sclerotium | 12 g |
| Gan Cao | Glycyrrhiza Uralensis, Radix | 3 g |
| He Huan Pi | Albizzia Julibrissin, Pericarpium | 15 g |
| Xu Duan | Dipsacus Asper, Radix | 12 g |
| Shou Di | Rehmannia Glutinosa, Radix | 15 g |
| Gou Qi Zi | Lycium chinensis, Fructus | 12 g |
| Chen Pi | Citrus Reticulata, Fruit Pericarp | 9 g |
| Fang Feng | Ledebouriella Seseloides, Radix | 9 g |
| Wu Ling Zhi | Trogopterus xanthipes | 9 g |

The last herb was added for unrelated symptoms (sore feet). The preceeding six herbs are in the subsets: He Huan Pi=subset ‘Endo with anxiety’, then Xu Duan=subset ‘Endo with lower backache’, Shou Di and Gou Qi Zi=subset ‘Endo with blood deficiency and poor immunity’, finally Chen Pi and Fang Feng=subset ‘Endo affecting the bowels’.

Dosage: 150 mls decoction morning and evening. The patient was also prescribed the same Endometriosis tincture, to be taken when the decocted herbs were finished., 2 mls in water, AM and PM.

Third and Last Consultation

At the third consultation five weeks later, the patient reported that her condition was very good, ‘even under tremendous stress’ (due to her job). She had run out of herbs two weeks previously, and continued with the Endometriosis tincture. At her period three days before this consultation, there was brief mild abdominal pain, very little clotting, and the menstrual colouration was normal. The lower back was not painful, and the bowel movements unremarkable.

In order to consolidate her progress, maintain her immunity, and assist her coping abilities in the face of her intense stress, she was given a prescription to take over the next two months, with instructions to return should she experience any more discomfort.

Prescription 3

| Chinese name | Pharmaceutical name | Herb dosage |
|---|---|---|
| Chi Shao | Paeonia Obovata, Radix | 12 g |
| Bai Shao | Paeonia Lactiflora, Radix | 12 g |
| Dang Gui | Angelica Polymorpha, Radix | 12 g |
| Xiang Fu | Cyperus Rotundus, Rhizoma | 12 g |
| Yan Hu Suo | Corydalis Turtschaninovii, Rhizoma | 12 g |
| Bai Zhu | Atractylodis Macrocephalae, Rhizoma | 12 g |
| Gan Cao | Glycyrrhiza Uralensis, Radix | 3 g |
| He Huan Pi | Albizzia Julibrissin, Pericarpium | 15 g |
| Ye Jiao Teng | Polygonum Multiflorum, vine stem | 30 g |
| Gou Qi Zi | Lycium chinensis, Fructus | 12 g |
| Chen Pi | Citrus Reticulata, Fruit Pericarp | 6 g |
| Fang Feng | Ledebouriella Seseloides, Radix | 9 g |
| Dang Shen | Codonopsis Pilosa, Radix | 12 g |
| Zhi Ke | Citrus aurantium, Fruit pericarp | 15 g |

The last two herbs were added to assist the patient's energy and coping abilities. The proceeding five herbs are in the subsets: He Huan Pi and Ye Jiao Teng=subset ‘Endo with anxiety’, Gou Qi Zi=subset ‘Endo with blood deficiency and poor immunity and Chen Pi and Fang Feng=subset ‘Endo affecting the bowels’. Dosage: 150 mls decoction morning and evening. The patient was also prescribed the same Endometriosis tincture, to be taken when not taking the decocted herbs, 2 mls in water, AM and PM.

### Exaple 11

### Case Study 7

Patient 3 was 29 years of age. Endometriosis had been diagnosed 3 years prior to presentation, by laparoscopy, which she had undergone due to rectal pain at menstruation. After that surgery, she had conceived and borne a child, following which the symptoms had gradually returned. She suffered from bilateral abdominal pain, and recently a reappearance of the rectal pain suggestive of endometriosis recurrence. She also had dyspareunia (painful intercourse) with deep vaginal discomfort regularly at midcycle: cyclic pain is another indicator of recurrence of endometriosis. Periods had become discoloured, clotted, heavy and extended since she ceased the contraceptive pill 3 months prior to presentation.

She also suffered from premenstrual mood swings, disturbed sleep, headaches, constipation and abdominal bloating. She was advised to take the following prescription:

Prescription 1

| Chinese name | Pharmaceutical name | Herb dosage |
|---|---|---|
| Bai Shao | Paeonia Lactiflora, Radix | 12 g |
| Dang Gui | Angelica Polymorpha, Radix | 12 g |
| Chuan Xiong | Ligusticum Wallichii, Radix | 9 g |
| Yan Hu Suo | Corydalis Turtschaninovii, Rhizoma | 9 g |
| Gan Cao | Glycyrrhiza Uralensis, Radix | 3 g |
| Chai Hu | Bupleurum Falcatum, Radix | 6 g |
| He Huan Pi | Albizzia Julibrissin, Pericarpium | 15 g |
| Sheng Di | Rehmannia Glutinosa, Radix | 15 g |
| Yu Jin | Curcuma Longa, Rhizoma | 9 g |
| Mu Dan Pi | Paeonia Moutan, Radix | 9 g |
| Shan Zhi Zi | Gardenia Jasminoides, Fructus | 12 g |
| Xia Ku Cao | Prunella vulgaris, Flos | 12 g |

The final four herbs were added to address the patient's auxiliary symptoms (i.e. Non-endometriosis related, such as headaches and digestive problems. The remaining herbs are in the preferred formulation or a subset.

Dosage: 150 mls decoction morning and evening.

Second Consultation

At the second consultation one month later, the patient reported that she had felt particularly good: “Give me more!’. There remained some ovulation pain in the right lower quadrant of the abdomen, but the vaginal discomfort had totally disappeared. The previous period had contained less clotting, and the colour and blood flow was normal.

She was advised to repeat the initial prescription without alteration.

Third Consultation

At the third consultation 9 years later, the patient reported that following the two months of herbal treatment, all pain, discomfort, and symptoms of endometriosis recurrence had disappeared for over 12 months, then very gradually returned over the next 4 to 5 years.

The present invention has been described with reference to specific preferred embodiments. It will be understood by those skilled in the art that variations in keeping with the principles and spirit of the invention are also encompassed herein.

1. A composition for treating or alleviating a symptom resulting from a gynecological disorder, comprising the herbs:

| | |
|---|---|
| Chi Shao | (Paeonia obovata, Root) |
| Dang Gui | (Angelica Polymorpha, Root) |
| Chuan Xiong | (Ligusticum Wallichii, Root) |
| Xiang Fu | (Cyperus Rotundus, Rhizome) |
| Yan Hu Suo | (Corydalis turtschaninovii, Rhizome) |
| Tao Ren | (Prunus persica, Seed), or parts thereof. |

2. The composition of claim 1, further comprising Poria cocos, Alisma plantago-aquatica, Atractylodes macrocephala, Spatholobus suberectus, Lindera Strychnofolia, Glycyrrhiza uralensis, or parts thereof.

3. A composition according to claim 1, wherein said composition is supplemented with one or more herbs selected from the group consisting of:

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit) |
| Fang Feng | (Ledebouriella seseloides, root) |
| Chen Pi | (Citrus reticulata, fruit pericarp) |
| Chai Hu | (Bupleurum falcatum, root) |
| Xu Duan | (Dipsacus asper, root) |
| Ye Jiao Teng | (Polygonum Multiflorum, vine stem) |
| Tu Si Zi | (Cuscuta, Seed) |
| Shan Zha | (Crataegus cuneata, fruit) |
| He Huan Pi | (Albizziae julibrissin, bark) |
| Yi Mu Cao | (Leonurus sibiricus, Herb) |
| Tian Qi | (Panax pseudoginseng, Root) |
| Hong Hua | (Carthamus tinctorius, Flos) |
| Sang Ji Sheng | (Loranthus parasiticus, Stem) |
| Xiao Hui Xiang | (Foeniculum Vulgare, Fruit) |
| Gui Zhi | (Cinnamomum cassia, stem), or parts thereof. |

4. A composition according to claim 3, wherein the composition is supplemented with the herbs:

| | |
|---|---|
| Bai Shao | (Paeonia lactiflora, root) |
| Sheng Di | (Rehmannia glutinosa, root) |
| Shou Di | (Rehmanniae glutinosae conquitae, root) |
| Gou Qi Zi | (Lycium chinensis, fruit). |

5. A composition according to claim 3, wherein the composition is supplemented with the herbs:

| | |
|---|---|
| Fang Feng | (Ledebouriella seseloides, root) |
| Chen Pi | (Citrus reticulata, fruit pericarp) |
| Shan Zha | (Crataegus cuneata, fruit). |

6. A composition according to claim 3, wherein the composition is supplemented with the herbs:

| | |
|---|---|
| Chai Hu | (Bupleurum falcatum, root) |
| Ye Jiao Teng | (Polygonum multiflorum, vine stem) |
| He Huan Pi | (Albizziae julibrissin, bark). |

7. A composition according to claim 3, wherein the composition is supplemented with the herbs:

| | |
|---|---|
| Xu Duan | (Dipsacus asper, root) |
| Tu Si Zi | (Cuscuta, Seed) |
| Sang Ji Sheng | (Loranthus parasiticus, Stem). |

8. A composition according to claim 3, wherein the composition is supplemented with the herbs:

| | |
|---|---|
| Yi Mu Cao | (Leonurus sibiricus, Herb) |
| Tian Qi | (Panax pseudoginseng, Root) |
| Hong Hua | (Carthamus tinctorius, Flos). |

9. A composition according to claim 3, wherein the composition is supplemented with the herbs:

| | |
|---|---|
| Xiao Hui Xiang | (Foeniculum Vulgare, Fruit) |
| Gui Zhi | (Cinnamomum cassia, stem). |

10. A composition according to claim 1, which includes extracts of the herbs.

11. A composition according to claim 1, wherein said composition is in the form of a powder, capsule, tablet, liquids or paste.

12. A composition according to claim 1, further comprising pharmaceutically acceptable extracts, adjuvents, solvents, carriers, flavours, colourings, or coatings.

13. A composition according to claim 1, wherein said composition is formulated with powdered herbs.

14. A composition according to claim 1, wherein said composition is in capsule or tablet dosage form.

15. A composition according to claim 1, wherein said composition is in a liquid or paste form.

16. A method for prophylactic or therapeutic treatment of a gynaecological gynecological disorder, comprising the step of administering to a subject requiring such treatment, a composition according to claim 1.

17. A method of alleviating or preventing a symptom resulting from a gynaccological gynecological disorder comprising the step of administering to a subject requiring such treatment, a composition according to claim 1.

18. A method according to claim 16, wherein said gynaecological gynecological disorder is endometriosis, adenomyosis, and/or dysmenorrhoea.

19. A method according to claim 18, wherein said gynecological disorder is endometriosis.

20. A method according to claim 18, wherein said gynecological disorder is adenomyosis.

21. A method according to claim 18, wherein said gynecological disorder is dysmenorrhoea.

22. A method according to claim 16, wherein said composition is administered orally or parenterally.

23. A method according to claim 22, wherein said composition is administered orally.

24. A method according to claim 16, wherein said composition is administered in a single bolus dose, multiple doses, or via a slow release device.

25. A method according to claim 16, further comprising the step of administering one or more therapeutic agents in addition to the composition.

26-34. (canceled)
